(19) United States
(12) Patent Application Publication
(10) Pub. No.: US 2013/0319890 A1
(43) Pub. Date: Dec. 5, 2013

(54) DISPOSABLE FEMININE HYGIENE ARTICLE

(71) Applicants: Frank Davis, Colorado Springs, CO (US); Iwona Davis, Colorado Springs, CO (US)

(72) Inventors: Frank Davis, Colorado Springs, CO (US); Iwona Davis, Colorado Springs, CO (US)

(21) Appl. No.: 13/910,116

(22) Filed: Jun. 5, 2013

Related U.S. Application Data
(60) Provisional application No. 61/655,762, filed on Jun. 5, 2012.

(51) Int. Cl. A61F 13/551

(52) U.S. Cl. CPC A61F 13/5518; A61F 13/55175
USPC 206/440; 53/473

(57) ABSTRACT

A disposable personal hygiene article includes a container, a bag inside the container, and a release mechanism. The container is attached to a personal hygiene article portion. During disposal, the bag is released from the container via the release mechanism and is configured to fit the personal hygiene article for disposal.

This application claims the benefits under 35 U.S.C. §119(e) of U.S. Provisional Patent Application No. 61/655,762 filed Jun. 5, 2012, which is hereby fully incorporated herein by reference.

### BACKGROUND OF THE INVENTION

### Field of the Invention

The present invention generally relates to disposable personal hygiene articles, and more particularly to a disposable feminine hygiene tampon, pad, and other similar articles.

### SUMMARY OF THE INVENTION

Accordingly, the invention is directed to disposable feminine or personal hygiene articles.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, a disposal mechanism for a personal hygiene article comprise a container comprising an opening, a bag fitted in the container, and a release mechanism attached to the bag and accessible outside the container, the release mechanism is operable to release the bag outside the container through the opening, wherein the container is attached to the personal hygiene article.

In another aspect of the present invention, a method for disposing a personal hygiene article comprises releasing a bag from a container, the container being attached to the personal hygiene article, and inserting a portion of the personal hygiene article inside the bag.

In yet another aspect of the present invention, a tampon package assembly comprises a tampon having a first and second end, a container attached to an area proximate to the first end of the tampon, the container comprising an opening, a bag fitted in the container, and a tab attached to the bag through the opening and accessible outside the container.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification. Illustrated embodiments of the invention and together with the description serve to explain the principles of the invention.

FIG. 1A illustrates an end view of the disposable personal hygiene article according to an embodiment of the invention;

FIG. 1B illustrates a side view of the disposable personal hygiene article of FIG. 1A;

FIG. 1C illustrates a top view of the disposable personal hygiene article of FIG. 1A;

FIG. 1D illustrates a side view of the disposable personal hygiene article after a bag is pulled from the container of FIG. 1A;

FIG. 1E illustrates a side view of the disposable personal hygiene article after the article is inserted into the bag of FIG. 1A;

FIG. 2 illustrates an exemplary disposable personal hygiene article according to another embodiment of the invention;

FIG. 3A illustrates an top view of the disposable personal hygiene article according to another embodiment of the invention;

FIG. 3B illustrates a side view of the disposable personal hygiene article of FIG. 3B;

FIG. 3C illustrates a cross-sectional view of the disposable personal hygiene article of FIG. 3A;

FIG. 3D illustrates a side view of the disposable personal hygiene article after a bag is partially pulled from the container of FIG. 3A;

FIG. 3E illustrates a side view of the disposable personal hygiene article after the article is partially inserted into the bag of FIG. 3A;

FIG. 3F illustrates an first end view of the disposable personal hygiene article after the article partially inserted into the bag of FIG. 3A;

FIG. 3G illustrates a second end view of the disposable personal hygiene article after the article is inserted into the bag of FIG. 3A; and

FIG. 3H illustrates a side view of the disposable personal hygiene article after the article is fully inserted into the bag of FIG. 3A.

### DETAILED DESCRIPTION OF THE ILLUSTRATED EMBODIMENTS

This invention relates to disposable personal hygiene articles.

A container which is used in embodiments of the invention may be a fixed storage medium for containing objects. Preferably, the container has an opening that allows objects to be conveniently removed or inserted. The container can be attached to a host article for convenient access of objects stored in the container to be used on the host article. The container can take various sizes and shapes to accommodate the objects to be fitted, the release mechanism to remove such objects from the container, and the host article of which the objects is used upon. In a preferred embodiment of the invention, the container is shaped and sized to attach to a tampon, pad, or other personal hygiene articles and to store a bag sized to store the article while being non-obtrusive to the hygienic uses of such article and the comfort of a user using the article.

A bag which is used in embodiments of the invention may be a rigid or non-rigid container for containing objects. The bag itself may be collapsible for storage in another container. In a preferred embodiment of the invention, the bag is sized to entirely store a personal hygiene article in the opened state but can be entirely stored within the container in a collapsed state. The bag may be of various colors, patterned, and/or may have other designs or shapes. The bag may be biodegradable, perfumed or scented, and/or may have other properties.

A release mechanism is used in embodiments of the invention may be a mechanism which aids a user in removing a bag from a container. In a preferred embodiment of the invention, the release mechanism includes a tab attached to the bag and accessible outside the opening of the container. A user can pull the bag out of the container by pulling on the tab. However, the release mechanism does not need to be a separate component. For example, the release mechanism can consist of the bag being pre-folded a certain way in the container or having an embedded perforation such that a user can release the bag from the container by pulling the bag directly through the opening or perforation.

One embodiment is directed towards a disposal apparatus for a personal hygiene article. The apparatus includes a container having an opening and a bag fitted in the container. A release mechanism attached to the bag and accessible outside the container, the release mechanism is operable to release the bag outside the container through the opening, wherein the container is attached to the personal hygiene article.

The release mechanism may be any type of mechanism including a tab, a string, a wire, and combinations of the same. The release mechanism may be dimensioned to be the same as or larger than the opening or it may be dimensioned to be smaller than the opening. The bag may be dimensioned larger than the personal hygiene article and/or the bag could be dimensioned to contain the personal hygiene article.

The bag may be constructed from a plastic material, thermoplastic material, paper material, recycled material, biodegradable material, and combinations of the same. The bag may also be scented and/or have any color or shading including any of the primary colors or combinations of the same, e.g., pink, black, and patterned. Optionally, the backside of the container opposite to the opening is attached to the personal hygiene article.

In one embodiment, the backside of the container opposite to the opening is attached to the personal hygiene article and the bag is dimensioned and operable to contain the personal hygiene article, wherein the release mechanism comprises a tab operable to release the bag by pulling, and wherein an opening of the bag is operable to be tied off after containing the personal hygiene article.

One embodiment, is directed towards a method for disposing a personal hygiene article. The method includes a user releasing a bag from a container, the container being attached to or integral with the personal hygiene article. The user can insert a portion of the personal hygiene article or the entire article inside the bag. The bag may also be configured with a sealing mechanism, e.g., string, channel locking seal or other type of sealing mechanism. Another embodiment is directed to a tampon apparatus in including a tampon having a first and second end. A container attached to an area proximate to the first end of the tampon or integral with a first end portion of the tampon. The container including an opening and the bag is arranged in the container. A tab or other mechanism is attached to the bag through the opening and accessible outside the container.

In order to more fully appreciate the present disclosure and to provide additional related features, the following references are incorporated herein by reference in the their entirety:

(1) U.S. Pat. No. 5,133,457 by Kadel, which discloses a tampon applicator assembly is disclosed wherein the protective wrap which initially packages the tampon applicator provides a simplified covering for disposal of the spent applicator. The tampon applicator wrap is opened at a pre-determined location such that a discrete portion remains which is significantly longer than the applicator barrel member. Accordingly, after tampon ejection, with the plunger telescopingly located within the barrel, that unit is then inserted within the discrete wrap portion, which includes a substantial length extending beyond the reinserted applicator. That substantial length is then twisted and tucked into the opened end of the applicator to maintain closure of the wrap for applicator disposal.

(2) U.S. Pat. No. 5,988,386 by Morrow, which discloses a feminine hygiene storage unit includes a tampon section and a plunger section. Both sections are generally cylindrical and are coaxial to one another. The tampon section has a plunger end, an expulsion end, and a tampon compartment. The tampon compartment contains a tampon. The plunger section has a tampon end, a distal end, and at least one plunger compartment. The plunger section has a slightly smaller diameter than the tampon section. The plunger section is adapted to slide into the tampon section, expelling the tampon. The plunger compartment is adapted to contain a variety of menstrual products, such as a panty liner, a sanitary napkin, a moistened towelette, a tampon disposal bag, or a pharmaceutical composition for relief of menstrual symptoms.

(3) U.S. Pat. No. 6,177,608 by Weinstrauch, which discloses a tampon for feminine hygiene having an absorbent core and a cover disposed around the core which is permeable to body fluids, the cover being provided with flexible barrier strips which are spreadable from the tampon surface. The barrier strips serve to close the space between the tampon surface and the walls of the vaginal duct when the tampon is inserted, preventing leakage especially in the time just after insertion. The barrier strips may be of either hydrophilic or hydrophobic material. In the embodiment in which the barrier strips are hydrophilic, they serve as absorbent elements of the tampon in addition to the tampon core. In the embodiment in which the barrier strips are of a hydrophobic material, they direct menstrual fluids inwardly to the absorbent tampon core. In yet another embodiment, the barrier strips may be of a folded two-layer construction filled with an absorbent material.

(4) U.S. Pat. No. 6,610,037 by Rosengrant which discloses, a disposal cover comprising a single sheet of flexible, fluid-impervious material with a tape fastening system applied to the sheets perimeters is disclosed. In the preferred embodiment, the sheet has two outwardly curved sides which taper toward two opposing ends. In use, a used sanitary item, either a pad or a tampon, is placed longitudinally in the center of the cover, and the curved sides and tapered ends are folded until they overlap, and then are fastened together with a tape fastening system. This results in the used sanitary item being completely concealed in a compact, sanitary package that can be easily and discreetly disposed of through the solid waste system.

(5) U.S. Pat. No. 6,773,423 by Osborn, III et al., which discloses a catamenial tampon in the form of a soft, conformable, hollow bag which is capable of being inserted digitally or through the use of a unique applicator. The tampon has an outer surface, an inner surface, a head portion and a trailing portion. The inner surface of the tampon defines an interior of the tampon wherein the head portion is closed and the trailing portion defines an opening into the interior of the tampon. The tampon may have a shape modulus of compression of less than 0.05 pounds force. The tampon preferably includes a removal string, which can be attached to the inside or outside surface of the tampon.

(6) U.S. Pat. No. 7,238,173 by Dobbs, which discloses a tampon disposal system for efficiently and sanitarily removing and disposing of a soiled tampon. The tampon disposal system includes a housing having at least one wall, an inner cavity, a plurality of perforated edges and a cleansing wipe that is enclosed within the housing. Removal of the perforated edges from the housing creates an opening along each perforation thereby exposing the inner cavity of the housing and the cleansing wipe positioned therein. Upon removal of the cleansing wipe, the string attached to a soiled tampon is insertable through the openings of the housing whereby removal and disposal of the tampon can be achieved in a relatively sanitary and efficient manner.

(7) U.S. Pat. No. 7,963,934 by Morrow, which discloses a tampon applicator assembly includes a tampon applicator section, an outer housing section, and a plunger section. The combined tampon applicator section and plunger section function as a conventional plunger-type tampon applicator. The outer housing section includes a sleeve with a proximal portion and a distal portion. The (distal) insertion end of the tampon applicator section essentially fits inside the proximal portion of the sleeve, and the distal portion of the sleeve is designed to accommodate a feminine hygiene product, such as a pad, a disposable wipe, a towelette disposal mitt, a simple disposal bag, and other products associated with menstruation.

Reference will now be made in detail to an embodiment of the present invention, example of which is illustrated in the accompanying drawings. Figures are shown for illustrative purposes and are not drawn to scale.

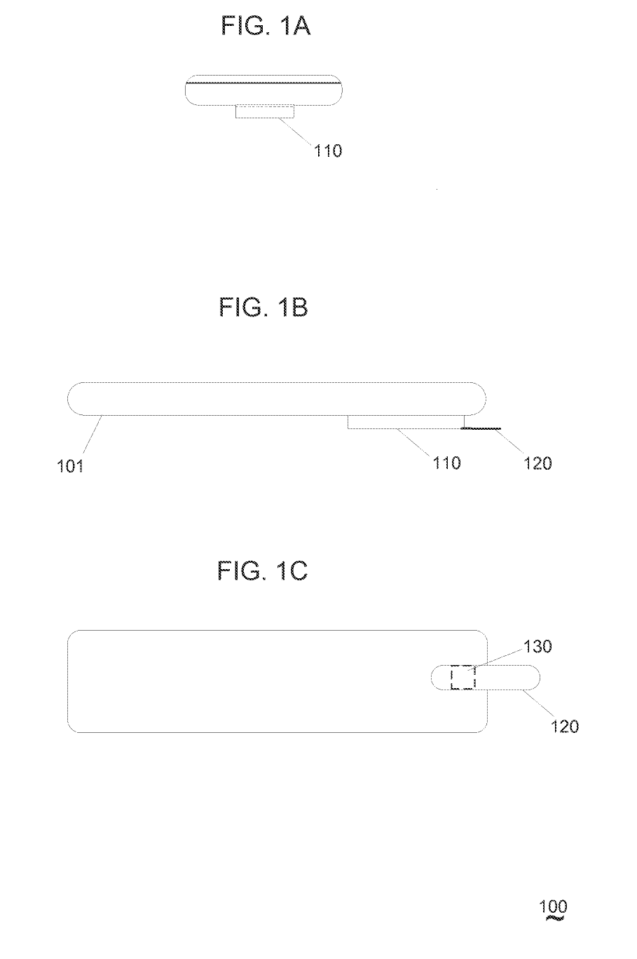

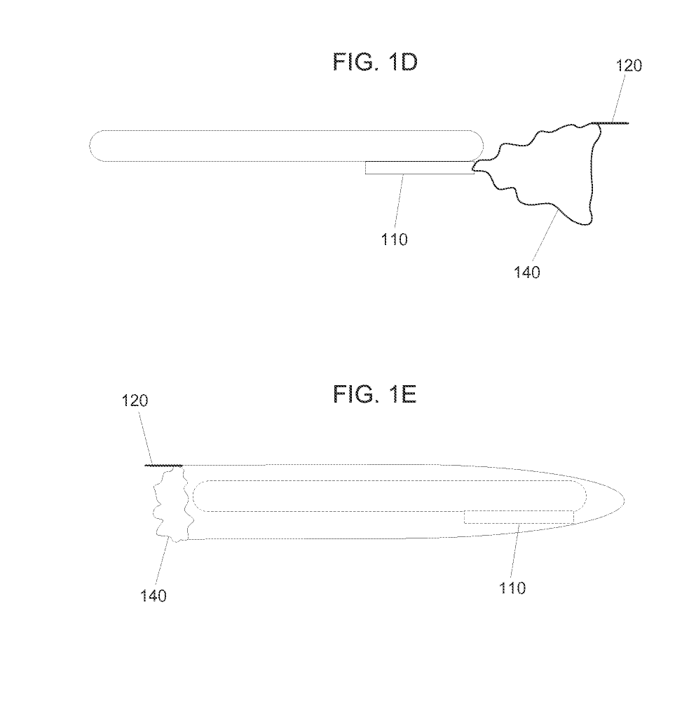

FIGS. 1A-E illustrate a disposable personal hygiene article, according to an exemplary embodiment. FIG. 1A illustrates an end view of the disposable personal hygiene article. FIG. 1B illustrates a side view of the disposable personal hygiene article. FIG. 1C illustrates a top view of the disposable personal hygiene article. FIG. 1D illustrates a side view of the disposable personal hygiene article after a bag is pulled from the container. FIG. 1E illustrates a side view of the disposable personal hygiene article after the article is inserted into the bag.

Referring to FIGS. 1A-E, disposable personal hygiene article 100 includes a tampon, pad, or other personal hygiene article portion 101, a container 110 with a hole or opening 130 attached to personal hygiene article portion 101, a bag 140 fitted inside container 110, and a release mechanism 120 attached to bag 140.

Prior to use by a user, disposable personal hygiene article 100 may be sealed and packaged by an external wrapping. During use, the external wrapping is removed, and personal hygiene article portion 101 is applied to the area of the user's body requiring hygiene attention. For this and other purposes, container 110 is attached to personal hygiene article portion 101 such that container 110 would be unobtrusive to the hygienic function of the article 100 and not uncomfortable for the user. For an article that is a tampon or pad, container 110 could be positioned proximate to one end of the article, and container 110 is shaped to contour the tampon or pad's shape. Moreover, the container may be an internal portion of the tampon or pad. The attachment can be made by adhesives or other means as known in the art. Moreover, the container may be integral to the personal hygiene article 100.

In a preferred embodiment, bag 140 is pre-fitted and housed within the container 110. When disposable personal hygiene article 100 is ready to be disposed, bag 140 is released from container 110 by release mechanism 120. Once bag 140 is released, the user can place the remainder of disposable personal hygiene article 100, which includes the soiled personal hygiene article portion 101 and attached container 110 into bag 140. Preferably, bag 140 is sized large enough to fit the entire remainder of disposable personal hygiene article 100. The opening end of bag 140 can further be sealed by tying, gluing, and other means. The opening end of bag 140 can be pre-fitted with tapes, seams, adhesives, or other joining agents to facilitate the sealing at disposal.

Release mechanism 120 facilitates the release of bag 140 from container 110. In a preferred embodiment, release mechanism 120 includes a tab accessible outside of container 110. The tab is connected to bag 140 through opening 130 of container 110. When the tab is pulled, bag 140 releases from container 110 as bag 140 is pulled through opening 130. In one configuration, release mechanism 120 is attached to container 110 via a perforation piece. Here, opening 130 is created when release mechanism 120 is pulled from container 110 and thereby ripping the perforated piece from container 110.

Bag 140 can have various colors, shapes, patterns, scents, and/or other properties. For example, bag 140 can be black to facilitate hiding the content inside bag 140 upon disposal. Alternatively, bag 140 can be pink or take other colors to be fashionable and more unique. Bag 140 can be scented with fragrances to facilitate masking unpleasant odors that might be associated with disposable personal hygiene article 100. Further bag 140 can be made of biodegradable material to facilitate processing after disposal. Properties for bag 140 in other configurations may also include being flushable.

In other configurations, other parts of disposable personal hygiene article 100 such as container 110 may also have properties such as being biodegradable and flushable to further facilitate processing after disposal.

In one embodiment of the invention, container 110 and bag 140 may be of one integral unit. For example, at least part of container 110 may be at least part of bag 140. In one configuration, container 110 is partially attached at an end portion to personal hygiene article portion 101. The end portion of container 110 is separate from the remaining portion of container 110 via a perforation. Further, the remaining portion of container 110 form bag 140. In this configuration, release mechanism 120 is the perforation separating the two portions of container 110. Bag 140 is released from the end portion of container 110 by the user tearing the perforation. In other configurations, container 110 and bag 140 is further compressed and attached as one integral unit with adhesives or other means.

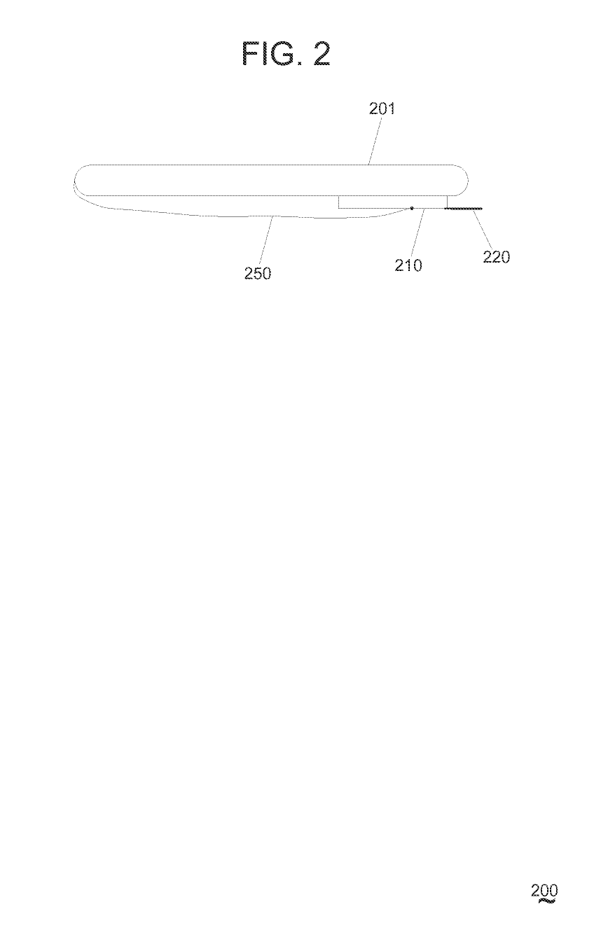

FIG. 2 illustrates a disposable personal hygiene article, according to another embodiment.

Referring to FIG. 2, disposable personal hygiene article 200 includes personal hygiene article portion 201, container 210 with an opening, release mechanism 220, and bagging mechanism 250. A bag is pre-fitted inside container 210.

During disposal, the pre-fitted bag is released from container 210 by release mechanism 220. Bagging mechanism 250 facilitates the bagging of the remaining disposable personal hygiene article 200 inside the bag without needing the user to unnecessarily make contact with soiled portions of personal hygiene article portion 201. For example, in one embodiment of the invention, bagging mechanism 250 is a string with one end attached to an attachment point of the bag and the other end attached to the one end of personal hygiene article portion 201 less proximate to container 210. When the bag is released from container 210, the string can be pulled at one convenient point with the bag lined up to cover personal hygiene article portion 201. Once the string is pulled, the bag is thereby consequentially pulled along through the entire covered personal hygiene article portion 201, thus obviating the need for the user to physically grab personal hygiene article portion 201 to insert it into the bag.

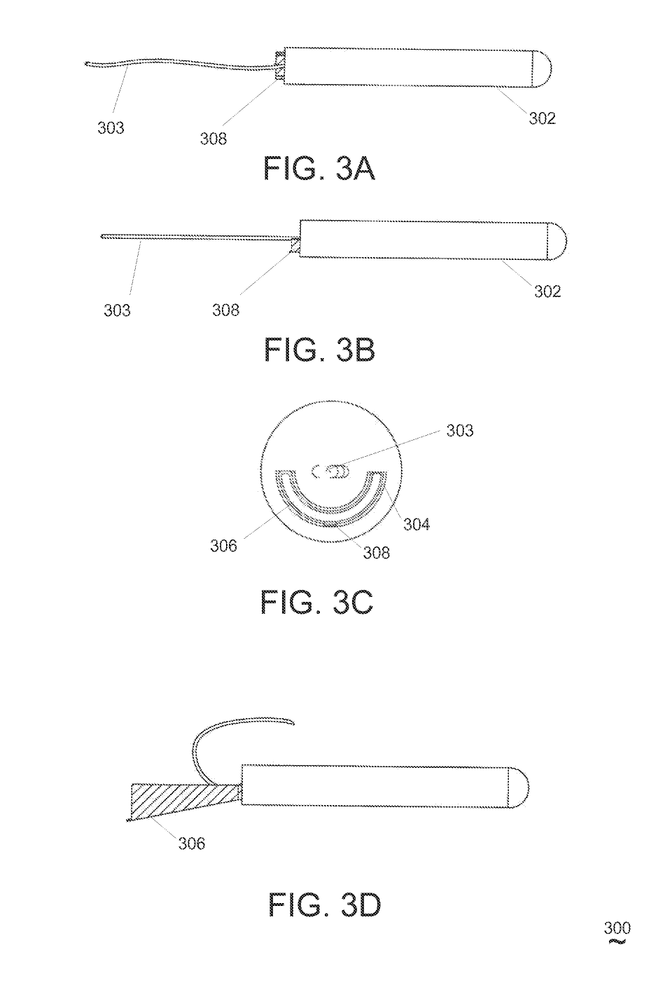

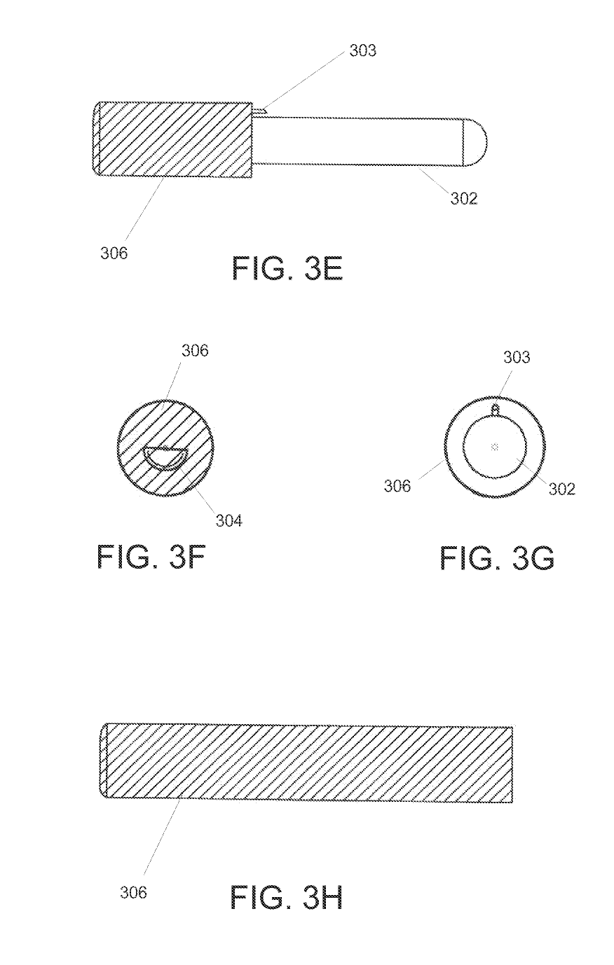

FIG. 3A illustrates a top view of the disposable personal hygiene article according to another embodiment of the invention. FIG. 3B illustrates a side view of the disposable personal hygiene article of FIG. 3B. FIG. 3C illustrates a cross-sectional view of the disposable personal hygiene article of FIG. 3A. FIG. 3D illustrates a side view of the disposable personal hygiene article after a bag is partially pulled from the container of FIG. 3A. FIG. 3E illustrates a side view of the disposable personal hygiene article after the article is partially inserted into the bag of FIG. 3A. FIG. 3F illustrates a first end view of the disposable personal hygiene article after the article partially inserted into the bag of FIG. 3A. FIG. 3G illustrates a second end view of the disposable personal hygiene article after the article is inserted into the bag of FIG. 3A. FIG. 3H illustrates a side view of the disposable personal hygiene article after the article is fully inserted into the bag of FIG. 3A.

Referring to FIGS. 3A-3H, a disposable personal hygiene article 300 includes a tampon, pad, or other personal hygiene article portion 302, a string portion 303, a container 304 integral to a personal hygiene article portion, a bag 306 fitted inside container 304, and a release mechanism 308 attached to bag 308.

Prior to use by a user, disposable personal hygiene article 302 may be sealed and packaged by an external wrapping (not shown). During use, the external wrapping is removed, and personal hygiene article portion 302 is applied to the area of the user's body requiring hygiene attention. For this and other purposes, container 304 is integral down an off-set lumen of the article portion 302 such that container 304 would be unobtrusive to the hygienic function of the article 302 and not uncomfortable for the user. For an article that is a tampon or pad, container 304 could also be positioned proximate to one end of the article, and container 304 and/or shaped to contour the tampon or pad's shape.

In a preferred embodiment, bag 306 is pre-fitted and housed within the container 304. When disposable personal hygiene article 302 is ready to be disposed, bag 306 is released from container 304 by release mechanism 308, e.g., tab, string, wire, and other mechanism. Once bag 308 is released, the user can place the remainder of disposable personal hygiene article 302, which includes the soiled personal hygiene article portion 302 and attached container 304 into bag 306. Preferably, bag 306 is sized large enough to fit the entire remainder of disposable personal hygiene article 302. The opening end of bag can further be sealed by tying, gluing, and other means. The opening end of bag can be pre-fitted with tapes, seams, adhesives, or other joining agents to facilitate the sealing at disposal.

Release mechanism 308 facilitates the release of bag from container 304. In a preferred embodiment, release mechanism 308 includes a tab accessible outside of container 304. The tab is connected to bag 306 through opening of container. When the tab is pulled, bag 306 releases from container 304 as bag 306 is pulled through opening. In one configuration, release mechanism 308 is attached to container via a perforation piece. Here, opening is created when release mechanism 308 is pulled from container 304 and thereby ripping the perforated piece from container.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

What is claimed is:

1. A disposal apparatus for a personal hygiene article, comprising:
a container comprising an opening;
a bag fitted in the container; and
a release mechanism attached to the bag and accessible outside the container, the release mechanism is operable to release the bag outside the container through the opening, wherein the container is attached to the personal hygiene article.

2. The disposal apparatus of claim 1, wherein the release mechanism comprises a tab, wherein the tab is operable to release the bag by pulling.

3. The disposal mechanism of claim 2, wherein the tab is dimensioned substantially same as or larger than the opening.

6. The disposal mechanism of claim 1, wherein the bag is dimensioned to contain the personal hygiene article.

7. The disposal mechanism of claim 1, wherein the bag mechanism is biodegradable.

8. A method for disposing a personal hygiene article, comprising:
releasing a bag from a container of the personal hygiene article; and
inserting a portion of the personal hygiene article inside the bag.

9. The method of claim 8, further comprising sealing an opening of the bag after inserting the personal hygiene article inside the bag.

10. The method of claim 8, wherein the bag is released through an opening in the container.

11. The method of claim 8, wherein releasing the bag from the container comprises pulling a tab accessible outside the container and attached to the bag.

12. The method of claim 8, wherein the tab is dimensioned substantially same as or larger than the opening.

13. The method of claim 11, wherein the tab is dimensioned smaller than the opening.

14. The method of claim 8, wherein the bag is dimensioned larger than the personal hygiene article.

15. A tampon package assembly, comprising:
a tampon having a first end, a second end and lumen extending from the first end;
a container formed at least in part from the lumen, the container comprising an opening;
a bag fitted in the container; and
a tab attached to the bag through the opening and accessible outside the container.

16. The tampon package assembly of claim 15, wherein the container is shaped to a contour of the tampon.

17. The tampon package assembly of claim 15, wherein the tab is dimensioned substantially same as or larger than the opening.

18. The tampon package assembly of claim 15, wherein the tab is dimensioned smaller than the opening.

19. The tampon package assembly of claim 15, wherein the opening is a slit.

20. The tampon package assembly of claim 15, wherein the bag includes one or more of a scented material and a color material.
